(19) United States
(12) Patent Application Publication
(10) Pub. No.: US 2015/0253226 A1
(43) Pub. Date: Sep. 10, 2015

(54) METHOD FOR SEPARATING CELLS-BEAD COMPLEXES

(71) Applicant: ACOUSORT AB, Lund (SE)

(72) Inventors: Per Augustsson, Lund (SE); Andreas Lenshof, Lund (SE); Thomas Laurell, Lund (SE); Stefan Scheding, Lund (SE)

(21) Appl. No.: 14/430,033

(22) PCT Filed: Sep. 20, 2013
(86) PCT No.: PCT/SE2013/051101

§ 371 Date: Mar. 20, 2015

Related U.S. Application Data
(60) Provisional application No. 61/708,169, filed on Oct. 1, 2012.

(30) Foreign Application Priority Data
Sep. 21, 2012 (SE) 1251064-0

(51) Int. Cl. G01N 1/40; G01N 33/569; G01N 33/543

(52) U.S. Cl. CPC G01N 1/4077; G01N 33/54313; G01N 33/56972; G01N 2001/4094

(57) ABSTRACT

The invention relates to a method and system to separate a subgroup of cells from a mixture of cells. This is achieved by creating a complex consisting of the subgroup of cells that are to be separated and a ligand that could be constructed of molecules such as antibodies, monoclonal antibodies, aptamers or fragments thereof specific for said subgroup of cells and microbeads present in a suspension. The suspension is then exposed to an acoustic field in one or two dimensions, which forces said cells and cell-bead complexes to separate from each other based on differences in size, density and compressibility.

### FIELD OF THE INVENTION

The invention relates to a method and system to separate a subgroup of cells from a mixture of cells. This is achieved by creating a complex consisting of the subgroup of cells that are to be separated and a ligand that could be constructed of molecules such as antibodies, monoclonal antibodies, aptamers, other antigen specific molecules or fragments thereof specific for said subgroup of cells and microbeads present in a suspension. The suspension is then exposed to an acoustic field in one or two dimensions, which forces said cells and cell-bead complexes to separate from each other when migrating across the interface between two properly chosen liquids which have a difference in density or compressibility.

### BACKGROUND

Affinity specific separations using microbeads in microfluidics system have emerged as a powerful means of targeted extraction of biological components in crude samples. This has since long been a standard in chromatography based systems, with packed microbead columns, for purification/enrichment of molecular species. Extraction of microbial and cellular targets from crude suspensions, however, have had limited success in packed bed based systems. A breakthrough for cell based separation along this line was the introduction of magnetic microbeads/nanobeads activated with antibodies against cell specific epitopes. In 1977 Molday et al. published a seminal paper where anti-mouse immunoglobuling activated magnetic nanoparticles (diameter □≈40 nm) bound to B-cells. The microbead coated B-cells were subsequently retained in a magnetic field generated by a horseshoe magnet while unlabeled cells were washed away [R. S. Molday, S. P. S. Yen and A. Rembaum, Nature, 1977, 268, 437-438.]. Improved extraction efficiency and process speed was obtained by passing the labeled cell solution past a dense network of ferromagnetic steel wire with the external magnet applied [R. S. Molday and L. L. Molday, Febs Letters, 1984, 170, 232-238.]. This provided locally stronger gradient field in the wire mesh and thus a higher retaining force on the labeled cells were obtained. This concept was further developed by Miltenyi [S. Miltenyi, W. Muller, W. Weichel and A. Radbruch, Cytometry, 1990, 11, 231-238.] and was commercialized by Miltenyi Biotech GmbH, providing new methods for affinity specific cell separation in preclinical and clinical preparatory applications of cell sorting and cell based therapy.

A further manifestation of such cell specific isolation is test-tube based magnetic affinity-bead extraction. A suspension containing cells is first incubated with microbeads coated with antibodies so that cell-bead complexes can form involving those cells that present on their surface the target molecules of the antibodies. Thereafter a magnet is placed close to the vessel containing the suspension whereby cell-bead complexes migrate towards the wall. The supernatant liquid is removed bringing along the non-targeted cells. The magnet is thereafter removed and the cell-bead complexes are re-suspended in new cell medium. (www.invitrogen.com/dynal)

Isolation of cells based on magnetic forces has two major drawbacks.

(1) The separation is strictly binary in the sense that if one or more beads are present on a cell, the cell will be captured by the magnetic field. Selective isolation of cells based on their expression level of certain surface molecule is therefore not feasible with current methods since they are not sensitive to the bead-load of the cells.

(2) Magnetic separation is well suited for batch processing wherein marked cells are trapped and wherein the trapping function has a limiting capacity. Continuous processing is difficult to achieve with magnetic forces since the magnetic beads will become trapped on the channel walls closest to the external magnetic field which causes a buildup of material in the flow channel that hinders the flow and prevents the targeted cell-bead complexes to be transferred.

Acoustophoresis, for which acoustically induced forces are utilized, provides a continuous process for the separation which is not limited by the volume of the sample. So far affinity-bead assisted extraction of cells has not been successful in acoustic separation systems. We propose herein that when introducing a second, properly chosen, liquid of different physical properties than the initial suspending liquid the discrimination of non-targeted cells increases vastly.

### SUMMARY OF THE INVENTION

The herein described method based on acoustic forces, acoustophoresis, circumvent the abovementioned drawbacks of magnetic extraction of cell-bead complexes from non-complexed cells by discrimination objects in liquid suspensions based on size, shape, density and compressibility in a miniaturized continuous flow format. Acoustophoresis has proven gentle to cells and does not affect cell viability or cell function. Previously acoustophoresis has been demonstrated for label-free sorting of cells into discrete fractions based on their individual physical properties. This invention relates to a novel manifestation of acoustophoresis where the bead-cell complexes are transferred from one suspending liquid into another, which has been modified to have different physical properties. The differences in properties between the two liquids causes the cells that are not complexed with beads to be immobilized in the interface between the liquids while bead-complexed cells continue further into the modified liquid. The number of beads, which reflects the expression level of the targeted surface molecule, dictates how far into the modified liquid the cell-bead complex will travel.

Acoustic standing wave force fields have been used extensively to focus/concentrate cells in microfluidic system, utilizing the fact that the flow channel serves as the fluid conduit at the same time as it acts as an acoustic resonator defined by the channel dimension. Efforts to separate different cells in acoustic force-fields based on their intrinsic acoustophysical parameters have been reported but have still suffered from the parabolic flow profile causing a variation in retention time in the force field performing the separation step.

The current invention discloses how a suspension of different groups of cells, such as white blood cells are mixed with a ligand such as an antibody/monoclonal antibody or fragment thereof and microbeads, wherein said ligand binds to a specific subgroup of cells, and the microbeads binds to said ligand forming cell-bead complexes. Pre-alignment of the cells and the cell-bead complexes may then be performed by designing a channel such that an acoustic standing wave field exerts forces on cells and cell-bead complexes in two dimensions. Pre-aligned cells and cell-bead complexes subsequently enter into a microchannel segment designed for an acoustic standing wave resonance mode, which yields a force field that performs the separation of the non-complexed cell and the cell-bead complexes along a single dimension. The pre-alignment of cells and cell-bead complexes ensures equal retention time in the separation zone, and hence problems commonly experienced in poor separation resolution due to the parabolic flow profile of laminar flow conditions are alleviated. Crucial for this invention is that during the separation, the cell-bead complexes migrate from one suspending liquid into cell/bead-free acoustic step gradient liquid (ASGL). Non-complexed cells are retained in the interface between the liquids due to their low migration rate in the ASGL. Microbeads have different physical properties, which mediates a higher migration rate in the ASGL and therefore the cell-bead complexes will migrate further into this liquid.

In one aspect the invention relates to a micro scale method for separating cell-bead complexes, comprising cells, ligands and beads from a mixture of different types of cells in a liquid suspension, comprising the steps of;
i) subjecting the suspension to pressure, wherein said pressure forces said suspension into at least one inlet (11) and into at least one pre-alignment channel present on a microfluidic chip,
ii) subjecting said suspension to a two dimensional acoustic force directed perpendicular to the length direction of the pre-alignment channel (15),
iii) forcing said mixture of cells and cell-bead complexes through the side branches of a trifurcated inlet while forcing cell free ASGL through the central branch of said trifurcated inlet (12) simultaneously into at least one separation channel, wherein said mixture of cells and cell-bead complexes are laminated towards the side walls of the channel,
iv) subjecting said cells and complexes to a one dimensional acoustic force directed perpendicular to the length direction towards the center of the separation channel (16) and
v) collecting cell-bead complexes at the end of the separation channel present on the microfluidic chip through at least one outlet (13).
vi) collecting non-complexed cells at the end of the separation channel present on the microfluidic chip through at least one outlet (14).

For the first time it is possible to separate cells having the same or similar characteristics by combining affinity specific coupling technology with the acoustic microfluidic technology and thereby be able to separate for example T-cells or from a population of white blood cells. This is achieved by forming complexes between specific subgroups of cells with ligands and microbeads and by introducing a two-dimensional acoustic force field in at least one pre-alignment microchannel where cells/particles can be directed into a defined position in the y-z-plane. This confines cell-bead complexes and cells to one or more discrete positions with a uniform flow velocity in the plane transverse to the flow, prior to entering the separation channel, hence making the separation deterministic such that the intrinsic properties of the cells and the fluid dynamic interaction between complexes and cells and the aqueous medium governs the separation outcome. The combined effect of pre-alignment and subsequent ASGL separation will thus increase the resolving power of such systems and enables for the first time the possibility to separate cell-bead complexes from other cells in a manner which is gentle, quantitative and which is well suited for upstream preconditioning and downstream analysis and treatment of acquired cells.

### BRIEF DESCRIPTION OF THE DRAWINGS

FIG. 1. Schematic of on-chip transport of two suspended species. Top view. Not to scale.

FIG. 2. Schematic of on-chip transport of two suspended species. Side cross-sectional view. Not to scale.

FIG. 3. Drawing suggested realization of the complete device. Top view. To scale.

FIG. 4. Drawing suggested realization of the complete device. Side cross sectional view as if cut in half along the vertical center plane. To scale.

FIG. 5. Schematic of the external fluidics driving the flows in the channel

FIG. 6. Schematic of the ASGL based separation principle.

### DETAILED DESCRIPTION OF THE INVENTION

Definitions

Letters (x), (y) and (z) refers to the spatial position along the length (l), width (w), and the height (h) of the microchannel, respectively. Letters (Qᵢ), (v), and (pᵢ) refers to volume flow rate, flow velocity and pressure, respectively where subscript (i) indicate multiple instances of a property.

The word suspension refers to a fluid containing solid particles or cells that are sufficiently large for sedimentation.

“Antibodies” as used herein includes polyclonal and monoclonal antibodies, chimeric, single chain, and humanized antibodies, as well as Fab fragments, including the products of a Fab or other immunoglobulin expression library. Antibodies, which consist essentially of pooled monoclonal antibodies with different epitopic specificities, as well as distinct monoclonal antibody preparations, are provided. Monoclonal antibodies are made by methods well known to those skilled in the art. The term antibody as used in this invention is meant to include intact molecules as well as fragments thereof, such as Fab and F(ab′).sub.2, Fv and SCA fragments which are capable of binding an epitopic determinant on a protein of interest. A Fab fragment consists of a mono-valent antigen-binding fragment of an antibody molecule, and can be produced by digestion of a whole antibody molecule with the enzyme papain, to yield a fragment consisting of an intact light chain and a portion of a heavy chain. A Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′).sub.2 fragment of an antibody can be obtained by treating a whole antibody molecule with the enzyme pepsin, without subsequent reduction. A (Fab′).sub.2 fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

Ligands are intended to mean an agent that connects the cells to the microbeads independent on how the connection is formed or maintained. The cell could have several ligands that are connected or bound to the cell as well as the microbead could have several ligands coupled/bound on the surface. The ligand gives an affinity specific coupling between two objects, such as the cells and the microbeads. Examples of ligands includes antibodies (as defined above), monoclonal antibodies, affibodies, aptamers, other antigen specific molecules or fragments thereof specific for a subgroup of cells and microbeads.

### Methods and System

The invention relates to micro scale method and systems for separating a subgroup of cells from a suspension, such as cells present in the blood.

### Sample Preparation

A suspension of cells, wherein said cells are a group of different cells, such as white blood cells are mixed with ligands (as defined above) specific for one group of cells and microbeads. The ligands are allowed to bind to one or more binding sites present on a subgroup of cells, such as epitopes on the cells, wherein one or more of the ligands may bind to one and the same cell. The microbeads are allowed to bind to the ligands either prior to the ligands binds to the cells or after.

In one embodiment the ligand is mixed with the microbeads and bound to the microbeads prior to that the ligand-microbead complex is further mixed with the cells. Thus the cells becomes affinity specific coupled to the ligand-microbead complex forming the cell-microbead complex.

The cells may be blood cells such as erythrocytes, platelets or leukocytes (such as: neutrophils, eosinophils, basophils, lymphocytes, monocytes, NK-cells and macrophages), as well as dendritic cells, stem cells/precursor cells, endothelial cells and epithelia cells. The beads may be polymer beads, magnetic beads, silica beads, metal beads. The size of the beads is smaller than the channel dimensions. Examples of antibodies are CD34 against hematopoietic stem cells, CD3, CD4 and CD8 against T-cells, CD19 and CD20 against B-cells, CD235a against erythrocytes, CD14 and CD33 against monocytes/macrophages, CD66b against granulocytes, CD11c and CD123 against dendritic cells, CD41, CD61 and CD62 against platelets, CD56 against NK-cells, CD236 against epithelial cells and CD146 against endothelial cells

Acoustic step gradient liquid (ASGL) Acoustic tailored liquid. Examples of ASGL includes Water, Ficoll, Percoll, Glycerol, PBS, Serum Albumine, Blood Plasma, NaCl as well as mixtures thereof. The density of ASGL being within 0 900-1.100 g/cm³, such as 1.000-1.077 g/cm³, 1.000-1.500 g/cm³, the compressibility of ASGL being within 2.10⁻¹⁰-1.10⁻⁹ Pa⁻¹, such as 5.10⁻¹⁰-7.10⁻¹⁰ and the viscosity of the ASGL being within 0.5 to 50 mPa.s, such as 1-50 mPa.s, 5-50 mPa.s, 5-40 mPa.s, 0,5-10 mPa.s and 0,5-5 mPa.s. The ASGL is important in this invention because it imposes a restriction on the objects, e.g. cells, when passing through the device. I.e. the physical properties of the cell-microbead complexes need to meet certain criteria regarding density and compressibility in order for them to be unlabeled cells.

### Method

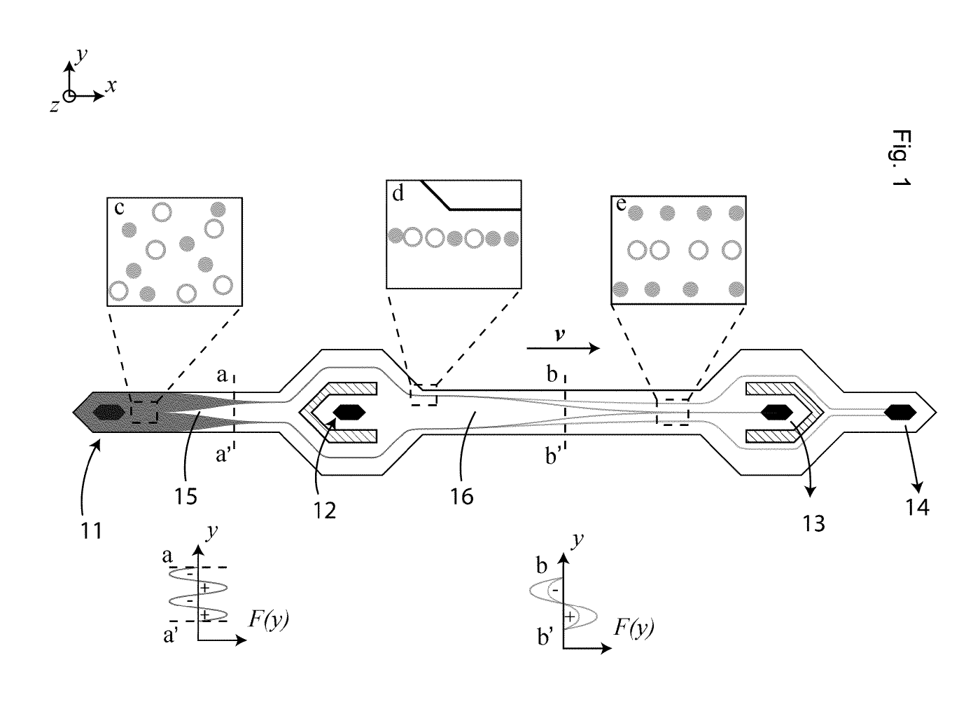

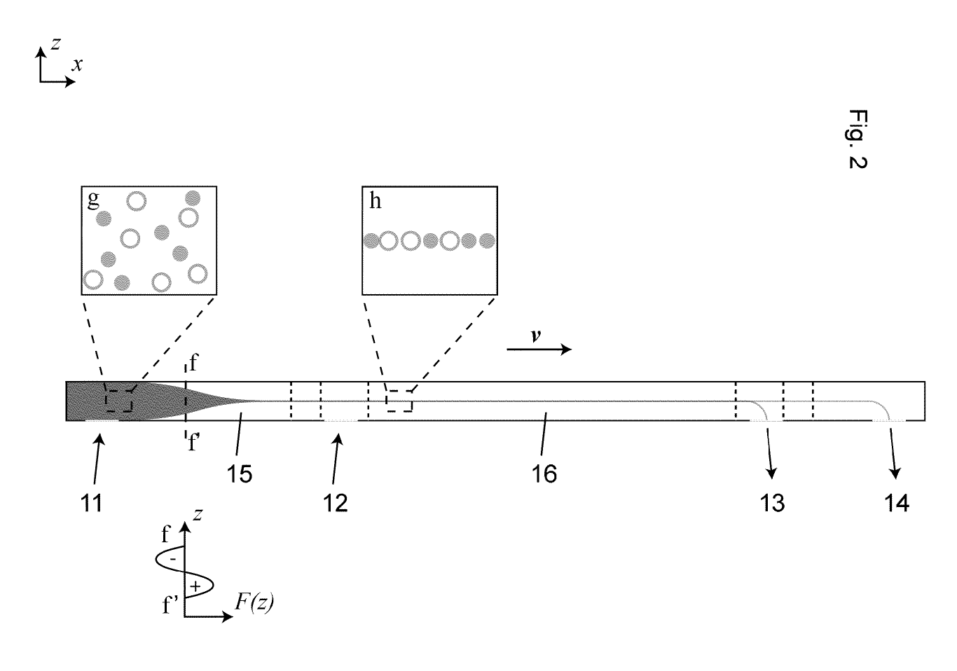

In one aspect the invention relates to a method, which comprises the steps of;
a) Forcing a suspension as defined above under sample preparation (51) via tubing (55) into at least one pre-alignment channel (15) by elevating the hydrostatic pressure pᵢ in the container of the suspension (51) such that the pressure at the channel inlet (11) is higher than the pressure at any of the other fluidic connections (12, 13, and 14). Said pressure difference cause a laminar flow in the channel that may be varied from 1 μL/min to 2 mL/min. The elevated pressure may be established using a syringe pump, a peristaltic pump, or by regulating the gas pressure in a sealed chamber wherein the container of the fluid is placed.
b) Subjecting the suspended cell and cell-microbead complexes (FIG. 1, inset c) in the suspension to a two-dimensional acoustic force potential (FIG. 1, a-a′ and FIG. 2, f-f) directed perpendicular to the pre-alignment channel (15), so that all of the cells and cell-bead complexes are localized into one or more points in the transverse cross-section of the flow. The two-dimensional acoustic force potential stems from resonant modes of ultrasound inside the water filled channel caused by reflections in the interface between the acoustically liquid and the acoustically hard walls of the channel. By tuning the frequency of the ultrasound vibrations, modes can be excited that forces suspended cells and complexes away from the walls, floor and ceiling of the channel towards the nearest vibrational pressure node. The constant influx of suspension at (11) in combination with the two-dimensional acoustic force potential causes the cells and cell-bead complexes of the suspension to align in narrow bands just before exiting the pre-alignment channel (FIG. 1, inset d and FIG. 2, inset h).

The pre-alignment channel may have a width and/or height ranging from 75 μm to 800 μm, such as from 75 μm to 200 μm, or ranging from 200 μm to 375 μm, or ranging from 300 μm to 400 μm, or ranging from 400 μm to 700 μm, or ranging from 700 μm to 800 μm, or being 150 μm, 300 μm, 188 μm, 375 μm or 750 μm.

The width and height of the pre-alignment channel may be related such that the width w divided by an integer number n equals the height h divided by an integer number m. In this case a single frequency of vibration may be chosen to fulfill a resonance condition simultaneously for the height and width dimension, such that

$$f = \frac{cn}{2 w} = \frac{c m}{2 h}$$

where c is the speed of sound in the suspending fluid. If the width and height of the channel are not related, the resonance condition may be controlled individually/selectively using separate frequencies of vibration for height and width, respectively. The frequency of vibration may vary in a range from 1 MHz to 10 MHz and is implicitly dictated by the dimensions of the channel as mentioned above and by choosing, either n=1, m=2, c=1500 m/s. Other examples are from 1-5 MHz, such as from 2-5 MHz or being 2 MHz or 5 MHz.

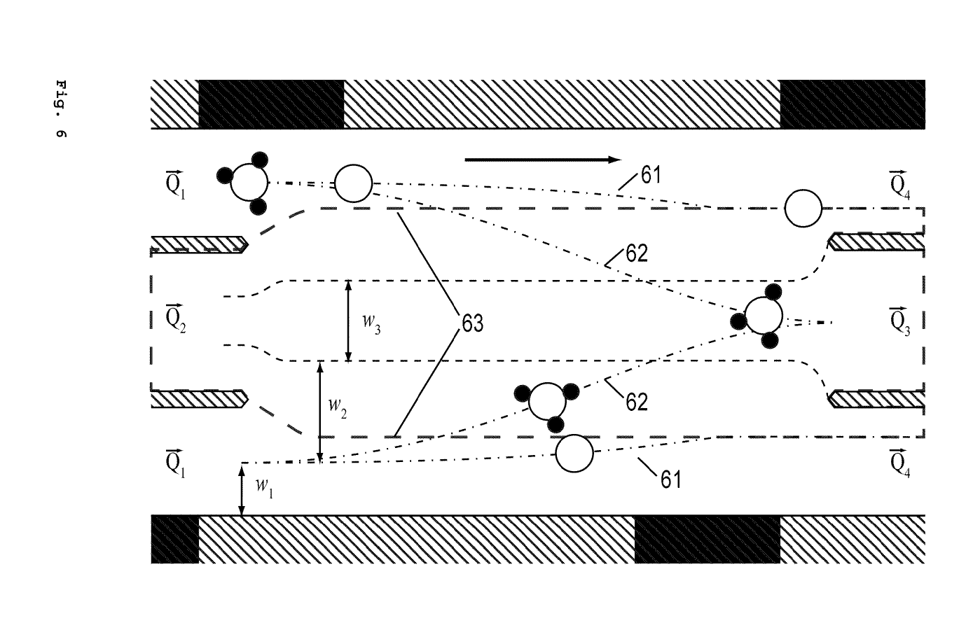

One example of this two-dimensional acoustic force potential is when a liquid filled pre-alignment channel (15) of width w₁₅=300 μm and height h=150 μm for a vibrational frequency f₁₅=5 MHz. For a stipulated speed of sound c in the suspension of 1500 m/s, the acoustic wavelength λ₁₅=C/f₁₅=300 μm. Given that the channel only can support multiples of half wavelengths it is clear that the vibration can induce resonance in the channel both along the y-axis (FIG. 1, a-a′) and along the z-axis (FIG. 2, f-f′). The combined yz-mode will have two vibrational pressure minima at (y₁, z₁)=(w₁₅/4, h/2) and (y₂, z₂)=(3 w₁₅/4, h/2).
c) Forcing said acoustically pre-focused suspended objects and infusing ASGL via a central inlet (12) simultaneously into a second separation channel (16), wherein said mixture of cells and/or cell-bead complexes are forced to proximity of one or more walls of said second channel. The suspension is thus laminated along one or both sides of an acoustophoresis microchannel while particle free liquid occupies the remaining part of the channel The relative volume flow rates Q₂ and Q₁, of the particle free liquid and the suspension, respectively, determine the lateral position (y=w₁) of the cells and/or particles, when entering the second channel (16). Increase of Q₂ leads to a decrease of w₁. The motive for doing so will be clear in paragraph (d and e).
d) Subjecting said mixture of cells and complexes to a one dimensional acoustic force potential directed primarily along the y-axis of said separation channel, so that all of the objects move towards the vertical center plane of that channel. The one dimensional acoustic force potential stems from a resonant mode of ultrasound inside the water filled channel caused by reflections in the interface between the acoustically soft water and the acoustically hard walls of the channel By tuning the frequency of the ultrasound vibrations, a half wavelength resonance mode can be excited that forces suspended objects away from the walls of the channel towards the vibrational pressure node in the channel center. The objects, that have previously been aligned by the 2-dimensional pre-alignment channel and are in proximity of the walls, move towards the vibrational pressure minima in the center of the channel (FIGS. 1 and 6)

When reaching the interface between their original suspending liquid and the ASGL the suspended cells and cell-bead complexes will not migrate further towards the center of the channel due the acoustic properties and/or the viscosity of the ASGL being different than those of the original suspending liquid. In order to propel a cell or cell-bead complex in an acoustic resonance the acoustic properties of the cell and cell-bead complexes and the suspending liquid must differ sufficiently. The composition of the ASGL can be chosen so that the transverse motion of non-complexed cells is kept at a minimum while cell-bead complexes can continue towards the vibrational pressure minima in the center of the channel This is possible due to the fact that the beads have different acoustic properties compared to the cells. Bead-bound cells can thus be dragged by the acoustic force exerted on the beads into the center of the channel, which mediates separation based on the molecular expression levels on the surface of the cells. (FIG. 6)

Cells that express high levels of for example an epitope will statistically bind more beads than cells having low expression levels. This bias in the number of beads contained by each cell-bead complex will cause the complexes to migrate to the center at different rates. By dividing the flow into different outlets at the end of the channel the cell-bead complexes can be sorted into discrete fractions based on their surface marker expression level profile.

The separation channel may have a width ranging from 75 μm to 800 μm, such as from 75 μm to 200 μm, or ranging from 200 μm to 375 μm, or ranging from 300 μm to 400 μm, or ranging from 400 μm to 700 μm, or ranging from 700 μm to 800 μm, or being 150 μm, 300 μm, 188 μm, 375 μm or 750 μm.

The width may be chosen such that the frequency of vibration f is

$$f = \frac{cn}{2 w}$$

where c is the speed of sound in the suspending fluid.

The frequency of vibration may vary in a range from 1 MHz to 10 MHz and is implicitly dictated by the specified dimensions of the channel as mentioned above and by choosing n=1 and c=1500 m/s.

One example of this one-dimensional acoustic force potential can be a liquid filled separation channel (16) of width w₁₅=375 μm and height h=150 μm for a frequency of vibration f₁₆=2 MHz. For a stipulated speed of sound c in the suspension of 1500 m/s, the acoustic wavelength λ₁₆=c/f₁₆=750 μm. Given that the channel only can support multiples of half wavelengths it is clear that the vibration only can induce resonance in the channel along the y-axis (FIG. 1, b-b′).

This mode will have one vibrational pressure minima at y=w₁₆/2.

Each cell and cell-bead complexes move in the x-direction along the channel driven by the flow, while being forced towards the central vibrational node, in the y-direction, at a rate that is determined by the acoustomechanical properties, mass density and compressibility of the suspending medium and the cell and cell-bead complexes, respectively. The rate is also determined by the size of the cell and cell-bead complexes and the strength of the acoustic resonance. By tuning the amplitude of the acoustic resonance in the second channel, the paths of the cells and cell-bead complexes may be deflected so that cells and complexes of dissimilar acoustic mobility exit the channel at different locations along the y-axis.

Since all cells and cell-bead complexes are pre-aligned in the yz-plane when entering the separation channel, their individual trajectories, reflect their acoustophysical properties and size, to a high degree. In absence of the pre-alignment the trajectories of individual cells and/or particles would be strongly influenced by their initial position when entering the separation channel, which is indeed not an intrinsic property of the cell and cell-bead complexes.
e) Adjusting the volume flow rates in the inlets (FIG. 6, Q₁ and Q₂) and outlets Q₃ and Q₄ to maximize the travel distance (w₂) at the cost of processing time. By imposing a longer travel distance for a cells and complexes to reach the central outlet (13), the number of faulty cells and/or particles in that outlet will be reduced. Decreasing Q₃ relative to Q₄ will narrow down the hydrodynamic aperture (w₃) of the central outlet and cause an increased (w₂). Similarly, decreasing Q₁ relative to Q₂ will diminish (w₁). The increased selectivity must be balanced against the demands regarding processing time of a sample of some finite volume, which is related to Q₁, and the ability of the external fluidics to produce a stable enough flow, which is related to Q₃ relative any disturbance in Q₄.

### System

In another aspect the invention relates to a system for separating a subgroup of cells and cell-bead complexes from a mixture of different types of cells and cell-bead complexes in a suspension, comprising;
i) a microchip having two inlets (11,12) and at least two outlets (13,14) and at least a first channel.
ii) means to provide pressure, wherein said pressure forces said suspension through an inlet (11) and into at least a pre-alignment channel (15),
iii) means to providing a two dimensional acoustic force (15),
iv) means to introduce a ASGL (defined above) into said channel (12)
v) means for providing a one dimensional acoustic force (16) and
vi) means for collecting objects having the same size and/or mass density and/or compressibility from at least two outlets (13,14).

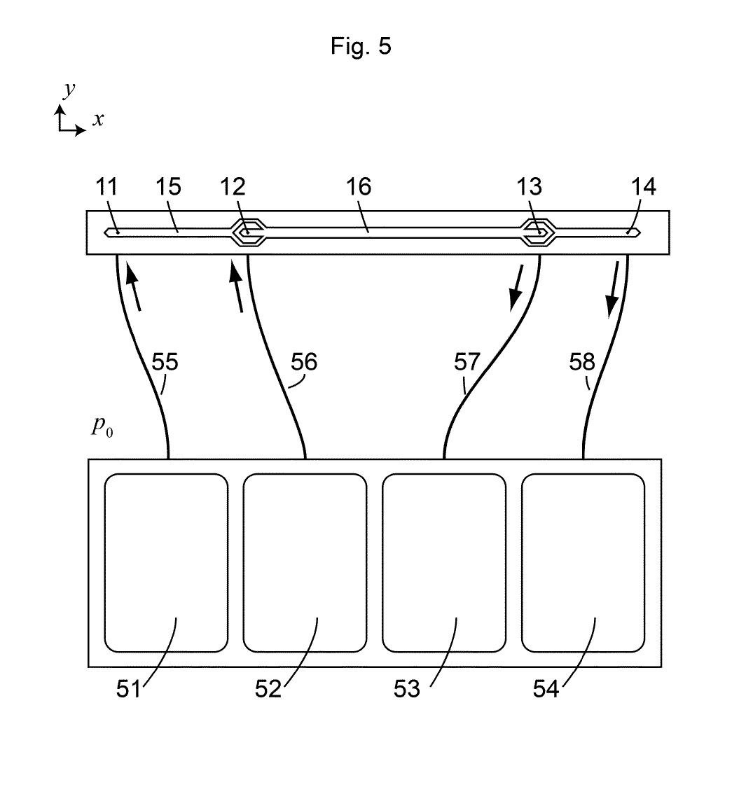

One example of a system is illustrated in the FIGS. 1-5. FIGS. 1 and 2 shows how the suspension comprising cells and cell-bead complexes are forced into the system (11). The suspension with the cells and cell-bead complexes (FIG. 1, insert c) is then transported through the pre-alignment channel (15) and exposed to a first two-dimensional acoustic force potential acting primarily in the yz-plane (FIG. 1, a-a′ and FIG. 2, f-f′, respectively) and the cells and/or cell-bead complexes are focused into two spatially separated flow streams. The two streams are then guided into two separate channels and ASGL is introduced into the system through (12). The ASGL forces the two pre-aligned streams of cells and cell-bead complexes out to positions proximal to the walls of the channel (FIG. 1, inset d). The cells and cell-bead complexes are then exposed to a one-dimensional acoustic force (FIG. 1, b-b′) (16), which separates cells and the cell-bead complexes from each other (FIG. 1, inset e). Cell-bead complexes are collected at (13) while non-complexed cells are collected at (14). The same can be seen in FIGS. 5, which is a simplified picture of what happens in the system upon use.

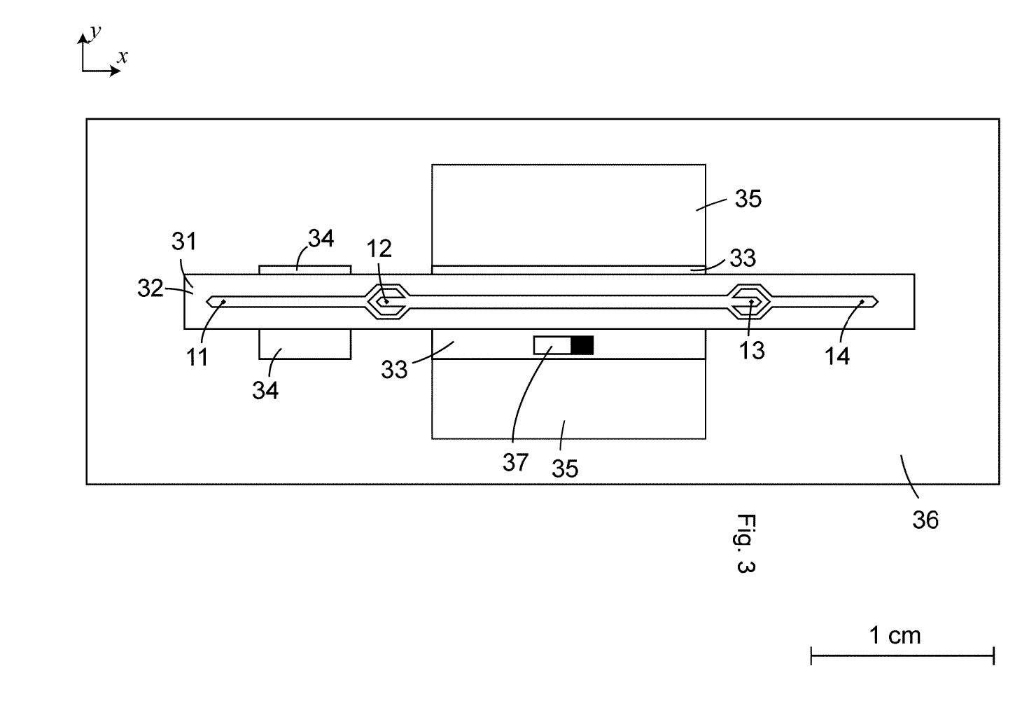

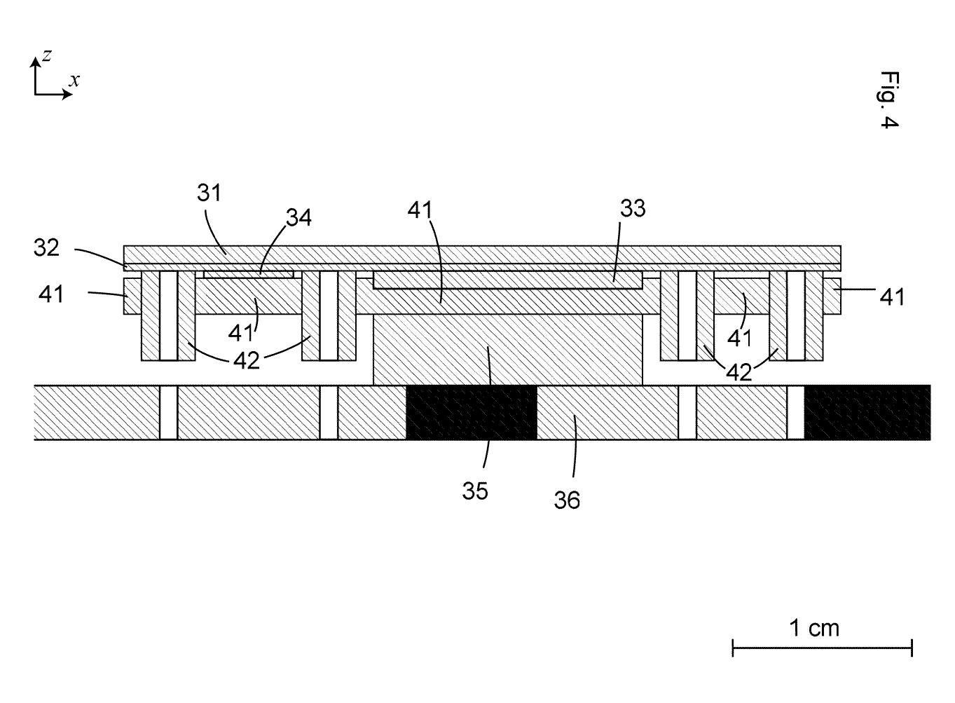

In one embodiment the microchip may be mounted as shown in FIGS. 3 and 4, which are two views of the same microchip.

In FIG. 3 there is an inlet (11) for the cells and cell-bead complexes in a suspension to enter the microchip. There is an inlet for the ASGL (12) and there are at least two outlets, one central (13) and one side outlet (14), where the cells and cell-bead complexes are collected.

Over the microchip with the channel there is a glass cover/ceiling to seal the microchannel (31). There is also silicon substrate (32) wherein the channel segments are etched. There is a transducer (33) for actuation of ultrasound in separation chamber. There is also a second transducer 2 present (34) for actuation of ultrasound for two-dimensional pre-alignment. In this embodiment there is also a Peltier element (35) present to be able to control the temperature in the device. In addition, is an aluminum microscope mounting plate/heat sink (36) present, to absorb excess heat from the Peltier element. A PT-100 thermo resistive temperature sensor (37) is used to monitor temperature for a feedback control loop.

In FIG. 4 an aluminum plate (41) is used for even distribution of heat in the device. There are inlet connectors (42), pieces of silicone tubing, the other references are as shown in FIG. 3 and explained above.

In FIG. 5, being a simplified overview of the system in operation. There is a pressure chamber for input cell and/or particle suspension (51), wherein the cells and the cell-bead complexes are forced into the microchip. There is one pressure chamber for ASGL(52), which forces the solution into the microchip. There are also two pressure chambers for the central outlet (53) and the sides' outlet (54). There are also four containers present in the chambers, one for the cell and our particle suspension (51), one for the ASGL (52), that may be any solution such as those mentioned above, one container for the central outlet (53) and one for the sides' outlet (54). There are also a number of tubings (55, 56, 57 and 58) that allows the transfer of the liquids from the chambers (51) and (52) and the two chambers (53) and (54).

In one embodiment of the invention the acoustic separation system is operated under iso-thermal conditions since thermal fluctuation may in some embodiments severely influences the acoustic properties of the microchannel acoustic resonators and hence the separation outcome. This may be realized by mounting the microfluidic acoustic separation chip on a Peltier-element that is computer controlled via a temperature sensor feedback at the separation chip vicinity. Thereby the temperature over the whole microchip is maintained at the same level. If the temperature is too high it might influence the cells and/or the particles. One example being when cells are to be separated that later are to be transferred into a mammal, such as a human being. If the cells are exposed to too high temperatures they get stressed or may die and cannot be transferred back to the subject in need of those cells.

The size of the microchannel constitutes an upper limit of the size of the cells and/or particles to be separated. The cells and/or particles to be separated may vary in shape and size ranging from 1 μm to 50 μm, such as 1-5 μm, 1-25 μm, 5-50 μm, 5-40 μm, 5-30 μm, 5-25 μm, 8-25 μm, or 8, 9, 10, 11, 12, 13, 14, 15, 16, 17, 18, 19 or 20 μm, or from 10-20 μm or 10-15 μm. The cells or particles may have a volume ranging from 0.0005 to 70×10⁻¹⁵ m³, such as 0.0005-0.003×10⁻¹⁵ m³, 0.0005-0.07×10⁻¹⁵ m³, 0.0005-8×10⁻¹⁵ m³, 0.05-0.10×10⁻¹⁵ m³, 0.07-70×10⁻¹⁵ m³, 0.07-35×10⁻¹⁵ m³, 0.07-14×10⁻¹⁵ m³, 0.07-8×10⁻¹⁵ m³, 0.25-6×10⁻¹⁵ m³0.3-8×10⁻¹⁵ m³, 0.07-35×10⁻¹⁵ m³ or 0.5-15×10⁻¹⁵ m³.

Following examples are intended to illustrate but not to limit the invention in any manner, shape, or form, either explicitly or implicitly.

### EXAMPLES

### Experimental Setting

A microchannel structure and holes for inlets and outlets was KOH etched in a <100> silicon wafer of thickness 350 μm and cut to the dimensions 40 mm by 3 mm (32). A piece of borosilica glass (31) (40 mm by 3 mm by 1 mm) was anodically bonded to seal the channel Inlets and outlets comprises of pieces of silicone tubing (42) which are glued to the backside of the chip to connect tubing for external fluidics.

All parts of the chip assembly were glued together. From bottom and up sandwiched together: an aluminum mounting plate also serving as heat sink, a Peltier element (35) (15 mm by 15 mm), an aluminum bar (41) (40 mm by 3 mm by 2 mm) with holes drilled to match the radius of the silicone inlet/outlet tubing, two piezoceramic actuators, one 5 MHz transducer (34) (5 mm by 5 mm) positioned below the pre-alignment channel, and one 2 MHz transducer (33) (15 mm by 5 mm) placed under the separation channel, and the acoustophoresis chip, see FIGS. 3 and 4. A Pt100 thermo-resistive element for temperature measurement, was glued onto the 2 MHz piezoceramic actuator alongside the acoustophoresis chip, see FIG. 3.

The piezoceramic transducers are driven by two signal-generators equipped with signal power amplifiers. The acoustic resonances can be controlled by tuning the frequency and voltage over the transducers.

Cells/microparticles in suspension enter a first pre-focusing channel (15) at a flow rate of 50 or 70 μL min⁻¹. A 5 MHz resonance in the yz-plane focus particles in two narrow bands, see FIGS. 1 and 2. Clean buffer medium enters through (12) at a volume flow rate of 450 or 490 μL/min and divides the pre-aligned particles further so that the stream of particles are pushed close to the walls of a separation channel (16). A 1.94 MHz resonance along the y-coordinate only focuses the particles towards the channels vertical center plane. At the end of the separation channel the flow is split up in a trifurcation outlet (13 and 14). The central outlet (13) volume flow is set to 150 or 280 μL/min and the volume flow rate in the combined side's outlet (14) was 250 or 280 μL/min.

### Example 1

### Separation of T Cells from Mononuclear Cells

Sample preparation. Peripheral blood progenitor cell samples were obtained from consenting healthy stem cell donors and patients undergoing leukapheresis. Mononuclear cells were isolated by density centrifugation and cell were incubated with super-paramagnetic polystyrene beads (Dynabeads, Invitrogen, Dynal CD4 positive, 4.5 μm in diameter) according to the manufacturer's instructions For flow cytometry (FACS) analysis and functional studies the beads were released from the cells using DETACHaBEAD solution.

Flow system setup. The volume flow in the system is controlled by three 10 mL glass syringes (1010 TLL, Hamilton Bonaduz AG, Bonaduz, Switzerland) which are mounted on a syringe pump (neMESYS, Cetoni GmbH, Germany). Two syringes on the outlets are set in withdrawal mode for maintaining a volume flow Q₃=30 μL/min in the central outlet and Q4=60 μl/min in the combined side's outlet, respectively (see FIG. 6 for definition of Qᵢ). The flow rates of these two syringes dictate the total volume flow (Qₜₒₜₐₗ=90 μL/min) rate in the main channel

The third syringe, infuses cell-free liquid in the central inlet to the channel at a volume flow rate corresponding to 67% of the total flow rate (Q₂=60 μL/min) For separation of bead bound cells from the mononuclear cell mixture, the liquid in the third syringe has to be more dense than the cell suspension. For the density step was undiluted Ficoll (Ficoll Histopaque, 1.077g/cm3) used. This density step enable separation of the bead labeled cell from the cell mixture. If the denser liquid is not used, all cells would act similar in the sound field and no separation will be possible. The cell mixture was drawn into the side's inlet from the bottom of a test tube, at atmospheric pressure, at a flow rate of 33% of the total volume flow rate (Q₁=30 μL/min) dictated by the net flux created by the three syringes.

Analysis of acquired outlet fractions. After acoustophoresis the cell and bead suspensions in the central respectively side outlet fractions were treated with DETACHaBEAD solution to break the bead bond to the cells. Magnetic beads were then removed with a magnet. Cells were then centrifuged, supernatant was removed, cell pellet labeled with CD4 fluorescent antibodies that target T-cells, washed and enumerated by flow cytometry (FACS Canto II flow cytometer and the FACSDiva software, BD Biosciences).

Acoustophoresis cell separation procedure. The ultrasound actuation frequency was adjusted to approximately 4.68 MHz in the first two-dimensional pre-focusing channel and in the second, separation channel, a fundamental resonance of 1.92 MHz was used. The driving voltage to the pre-focusing transducer was approximately 5 Vpp (2-7 Vₚₚ) and to the main separation transducer approximately 3 Vₚₚ (1-5 Vₚₚ). Because the use of ASGL in the center inlet, the mononuclear cells will not move into the center part of the chip but the complex will. The amplitude of the standing wave is set so that the complex barely just enter the center outlet. By doing this careful balance, mononuclear cells are prevented to slip into the center outlet stream. The amount of T-cells in the center fraction was enriched to 90±7%, compared to approximately 20% in the original sample.

1. A micro scale method for separating cell-bead complexes comprising cells, ligands and beads from a mixture of different types of cells in a suspension, comprising the steps of;
i) subjecting a suspension to pressure, wherein said pressure forces said suspension into at least one inlet and into at least one pre-alignment channel present on a microfluidic chip,
ii) subjecting said suspension to a two dimensional acoustic force directed perpendicular to the length direction of the pre-alignment channel,
iii) forcing said mixture of cells cell-bead complexes through the side branches of a trifurcated inlet while forcing cell free ASGL through the central branch of said trifurcated inlet simultaneously into at least one separation channel, wherein said mixture of cells and cell-bead complexes are pushed by hydrodynamic forces towards the walls of the channel,
iv) subjecting said cells and complexes to a one dimensional acoustic force directed perpendicular to the length direction towards the center of the separation channel and
v) collecting cell-bead complexes at the end of the separation channel present on the microfluidic chip through at least one outlet.
vi) collecting non-complexed cells at the end of the separation channel present on the microfluidic chip through at least one outlet.

2. The method according to claim 1, wherein said ligand is antibodies, monoclonal antibodies, affibodies, aptamers, single chain antibodies, antigen specific molecules or fragments thereof.

3. The method according to claim 2, wherein said cells are selected from the group consisting erythrocytes, platelets or leukocytes dendritic cells, stem cells/precursor cells, endothelial cells and epithelia cells.

4. The method according to claim 1, wherein said beads are selected from the group consisting of the beads may be polymer beads, superparamagnetic beads, silica beads, metal beads.

5. The method according to claim 1, wherein said ASGL has a density between 0.900-1.100 g/cm³.

6. The method according to claim 1, wherein the two dimensional acoustic force in step ii) consists of ½ wavelength and 1 wavelength perpendicular to each other.

7. The method according to claim 1, wherein said one dimensional acoustic force in step iv) consists of ½ wavelength.

8. The method according to claim 1, wherein said two dimensional acoustic force is caused by a frequency of from 1 to 10 MHz.

9. The method according to claim 1, wherein said method is maintained at a constant temperature.

10. The method according to claim 1, wherein the height or the width of channel is from 75 um to 800 um.
